(19) United States
(12) Patent Application Publication
(10) Pub. No.: US 2020/0023092 A1
(43) Pub. Date: Jan. 23, 2020

(54) FRAGRANCE DISPERSION SYSTEM

(71) Applicant: David E. Wachob, New Hope, PA (US)

(72) Inventor: David E. Wachob, New Hope, PA (US)

(21) Appl. No.: 16/037,363

(22) Filed: Jul. 17, 2018

(51) Int. Cl. A61L 9/03

(52) U.S. Cl. CPC A61L 9/035; A61L 9/127; A61L 9/037

(57) ABSTRACT

In one embodiment, a system is disclosed for providing fragrance to an artwork. The system includes a fragrance dispersion system configured to disperse a fragrance. The fragrance dispersion system is configured to be positioned adjacent to, within, or on an artwork.

### BACKGROUND

Until now, artwork has primarily been a visual medium, where a visual rendering has been portrayed upon a surface using paints, pencils, inks, glazes, markers, photographic, or other means, to visually detail a real or abstract image to a viewer. Numerous surface materials can be used for such renderings, including canvas, linen, leather, paper, cardboard, wood, clay, metal, plaster, ceramic, glass, stone or a wide variety of other materials. The images are presented on the surface by painting, sketching, etching, glazing, lithography, kilns, photographic or other conventional means and methods. This process has been used for centuries, dating back to cave art, created 30,000+years ago.

### BRIEF SUMMARY OF THE INVENTION

The proposed invention provides for a whole new dimension to be added to artwork, primarily, although not exclusively, paintings, drawings and photography. It does so by providing the sense of smell to such artwork, through a variety of fragrance enhancements and delivery means and methods, described below.

### BRIEF DESCRIPTIONS OF THE DRAWINGS

FIG. 1 is a high level view of a fragrance dispersion system;

FIG. 2 is a schematic diagram of a more detailed view of a fragrance dispersion system;

FIG. 3 is a schematic diagram of another more detailed view of a fragrance dispersion system;

FIG. 4 is a schematic diagram of another more detailed view of a fragrance dispersion system;

FIG. 5 is a schematic diagram of an even more detailed view of a fragrance dispersion system;

FIG. 6 is a schematic diagram of a further detailed view of a fragrance dispersion system

### DETAILED DESCRIPTION OF THE INVENTION

While strikingly beautiful, the above methods only provide a visual portrayal of whatever image the artist is trying to convey in his or her artwork. Sight is but only one of the senses that the human body is capable of receiving. With the addition of the sense of smell to art, the experience of both the artist and the recipient observing the art will be greatly enhanced. As such, it is the intent of the proposed invention to add the dimension of fragrance to artwork.

Artwork or art, is defined in the broadest sense, and includes paintings, sketchings, etchings, glazing, lithography, photography, sculpturing, tapestries, etc. Artwork can include a single pieces of artwork, or multiple pieces of artwork. Artwork, unless otherwise stated, can also include framing around, behind, underneath or in front of, such as wood, metal, plastic, cardboard, composite, glass, etc., or stands, pedestals, or structures to support or affix the artwork.

Several means and methods to add fragrance(s) to artwork are detailed below, to create fragrance dispersion systems. They can be classified in 3 main embodiments, including adding fragrance(s) such as a fragrance additive to the painting/portrayal material(s) and/or base materials themselves of the artwork, adding one of several possible fragrance dispersion systems or apparatus to the fame, artwork, or structure supporting the artwork, or providing a supplement fragrance dispersion system separate from the frame, artwork or structure. The various alternatives for each means and methods will be discussed separately.

### First Embodiment

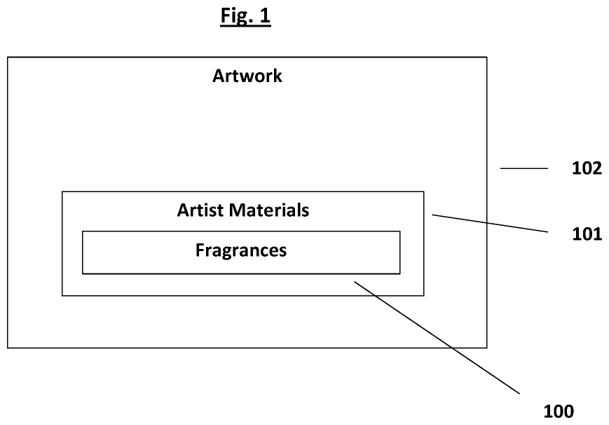

In the first embodiment of the invention detailed in FIG. 1, one or more fragrances 100 are added directly to one or more of the artist materials 101 used to create the painting, portrait, statue or otherwise artwork 102 to create the Fragrance Dispersion System, or “FDS”, containing fragrances 100 and artist materials 101 within the artwork 102. These materials 101 include not only the artist's creative materials such as paints (oil, acrylic, water colors, etc.), pencils, pens, charcoals, pastels, inks, dyes, glazes, waxes, clays, resins, shellacs, varnishes, concrete, markers, tiles, photographic, paper mache or other creative materials, etc., but could also include, additionally or separately, the artist's base materials themselves that the artwork is created on, in, or nearby, such as the canvas, linen, cloth, leather, plastic, fabric, paper, velum, glassine, plaster, ceramic, wood, metal, stone, clay, bronze, glass, etc., etc.

Any such fragrances 100 could be extracted from naturally occurring substances including, but not limited to flowers, fruits, woods or trees, leathers, foods (uncooked, cooking or already cooked), liquids, spices, nuts, vegetables, candy, etc., or created synthetically to imitate an existing fragrance, or to create a new fragrance. A wide variety of techniques for extraction of the above fragrance types can be used, including distillation (using water or alternative solvents), expression, infusing, reduction, enfleurage, pressing, heat and/or pressure extraction, and/or chemical or water extraction methods, using hexane solvent as but one example. Such processes could result in a wide variety of concentrated fragrance formats, including oils, extracts, tinctures, resinoids, pomades, concretes, absolutes, esters, terpenes, aldehydes, ketones, phenols, alcohols, hydrolats and oxides, etc.

Alternatively, “non-conventional” fragrances (or combinations thereof) could also be utilized either synthetically or naturally, to portray alternative and/or composite fragrances, such as spring meadow, autumn smells, burning wood, baby fresh, mildew, emotions, moods, fire, or any type of fragrance that the nose might be capable to detecting, good or bad, to add and portray a given fragrance.

The fragrances 100 could be very specific and generally known to all, such as a rose, for example, or much more subject to interpretation by the artist and/or the person who encounters the artwork 102, such as including a portrayal of the fragrance for happiness, love, relaxation, despair, darkness, or even death. This could be a single desired fragrance, or a multitude of fragrances, depending upon the simplicity or complexity of the desired fragrance(s) to be included.

Any of the desired fragrance(s) 100 can be added directly to the artist's creative or base materials 101 used to create the artwork 102. They can either be added by mixing or otherwise combining the fragrance(s) 100 into the materials either used to create the artwork itself (such as paint or glaze, as but one example) or the artist's base materials of the artwork (such as into/onto the canvas, linen or other fabrics, wood, paper, leather, cardboard, ceramic, plaster, cement, plastic, etc.), or both.

If the fragrance(s) 100 are distilled into an oil base, for example, then the fragrance(s) 100 could be easily combined with oil base paint(s). Alternative extractions, reductions or synthetic representations of the fragrance(s) 100 can also be added to oil or acrylic based paints or any other of the artist's creative or base materials described previously. Additionally, the fragrance(s) 100 could be added in and/or on coatings on and/or with or around the artwork (such as lacquers, sealants, clear coats, shellacs, varnishes, sprays, etc.), either before, during or after the artwork is completed. It may be desirable to optionally add a drying agent (not shown) with the fragrance(s) 100, to reduce the drying time when used with any paints or other artist's creative and/or base materials 101 due to the additional moisture or other compounds present in the fragrance(s) 100. Similarly, chemicals could also be added to assist in the combining of the fragrances and the artist's creative or base materials.

The fragrance(s) 100 can be included at the time of manufacture of the artist's creative and/or base materials 101, as desired before or during the production of the artwork, or even after the art has been produced. A wide variety of manual, chemical, pressure, heat, deposition, infusion, distillation, mixing or other means and methods are possible for inclusion of the fragrances 100 into the artist's creative or base materials 101.

The fragrance(s) 100, either separately or in conjunction with the artist's creative or base materials 101, could be universally applied to the artwork 102, or to location specific areas of the artwork 102. One such example of this could be including the desired fragrance for a flower when depicting the flower in the artwork, and using a different fragrance when depicting a tree in the artwork. All of the above fragrance dispersion systems become an integral part of the artist's artwork 102.

### Second Embodiment

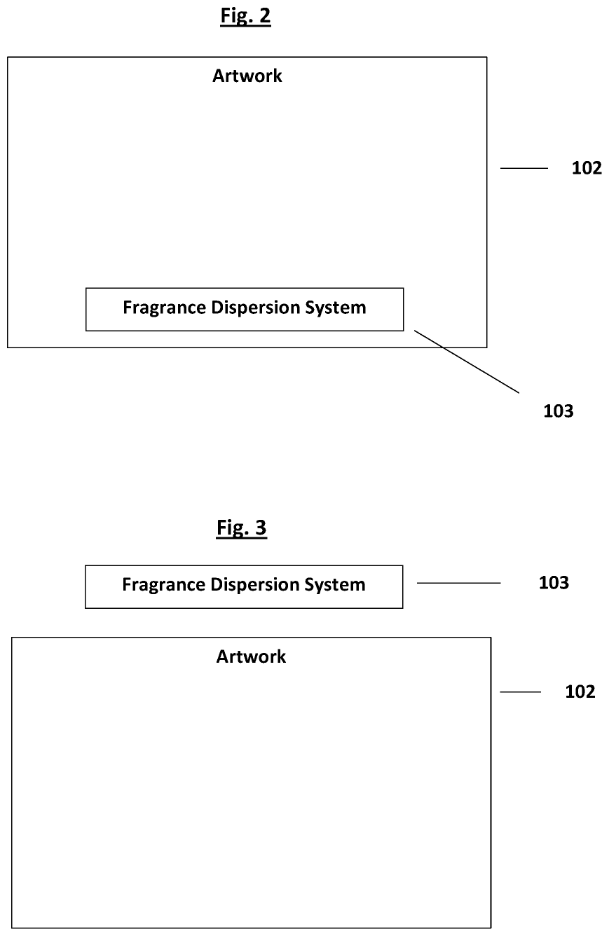

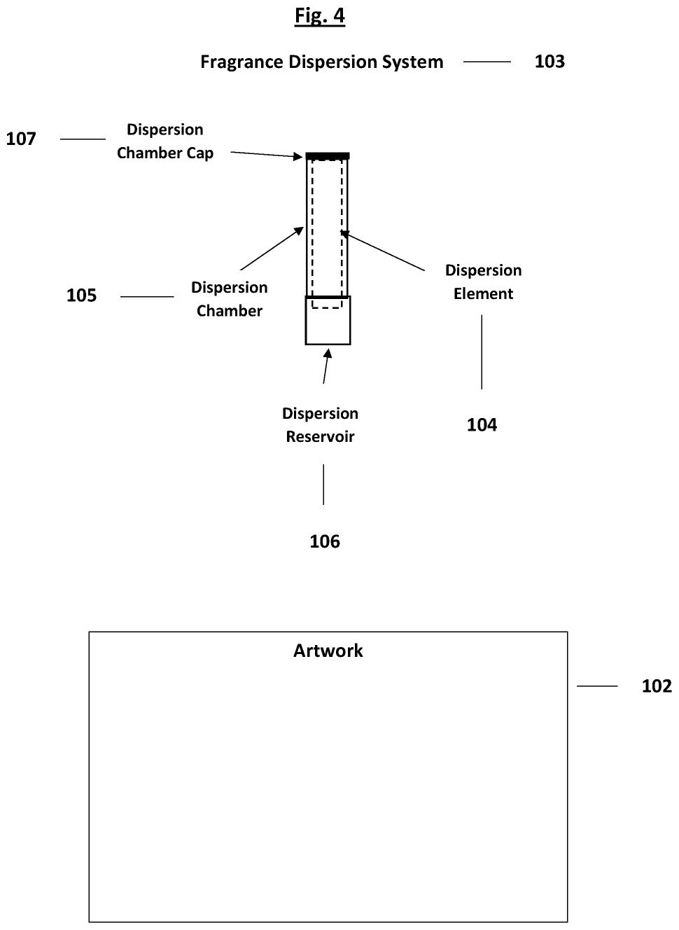

In the second embodiment of the invention, the Fragrance Dispersion System 103 is separately included as a part of (in FIG. 2) or supplemental to (in FIG. 3) the artist's artwork 102 and/or “frame” (including, but not limited to conventional framing (wood, composite, plastic, metal or otherwise), as well as non-conventional framing (such as platforms, stands, and/or support structures, viewing devices, etc.). A wide variety of Fragrance Dispersion System 103 variations are possible as described below.

The components of the second embodiment of the Fragrance Dispersion System 103 in FIG. 4 include: the Dispersion Element 104, the Dispersion Chamber 105, the Dispersion Reservoir 106 and the Dispersion Chamber Cap 107. The Dispersion Element 104 is required, while any or all of the Dispersion Chamber 105, Dispersion Reservoir 106 and the Dispersion Chamber Cap 107 are optional.

The Dispersion Element 104 is the component in the FDS 103 which holds and disperses the actual fragrance(s) 100. It can be constructed out of a wide variety of generally (although not exclusively) porous materials, such as a natural or synthetic wick, including cotton, wool, polyester, etc., or other material and/or formats, such as leather, cardboard, felt, cotton ball, pipe cleaner, wood, or composite material. The Dispersion Element 104 is shaped and sized accordingly to fit within the Dispersion Chamber 105, to optimize the dispersion of the fragrance(s) 100. It should be noted that “optimization” could include both slow and/or fast fragrance dispersion, depending upon the desired effect, such as the longest lasting FDS, or the most effective (such as most fragrant) FDS, or any combination thereof.

The Dispersion Chamber 105 can be fabricated out of a wide variety of materials, including metal, plastic, wood, card board, ceramic, clay, composites, etc. The Dispersion Chamber 105 could include one or more perforations, holes and/or other openings, to allow the fragrance(s) 100 to disperse outwards from the Dispersion Chamber 105, as well as providing for replenishment of the fragrance(s) 100 in the Dispersion Element 104 from the Dispersion Reservoir 106 (both detailed below).

Alternatively (or in conjunction with), the Dispersion Chamber 105 can be constructed out of porous material, to also allow the fragrance(s) 100 to disperse from the Dispersion Chamber 105 (as well as replenishment, described above). If the Dispersion Chamber 105 is sufficiently porous to allow the fragrance(s) to disperse from the Dispersion Chamber 105, then such Dispersion Chamber 105 could also serve as a second dispersion element, or replacing the previously mentioned Dispersion Element 104 altogether and therefore serving as the Dispersion Element 104 of the FDS 103.

The Dispersion Chamber 105 can be fabricated into any shape or size, such as cylindrical, rectangular, square, circular, or any other appropriate shape to interface to (or be included within, on, above, behind, under or adjacent to) the artwork. Additionally, although the orientation of the Dispersion Chamber 105 illustrated in FIG. 4 is “vertically illustrated”, it is only shown that way for convenience, and can be oriented in any possible direction.

The Distribution Reservoir 106 in FIG. 4 holds a given supply of the fragrance(s) 100 to be dispersed by the Dispersion Element 104, and as such, is connected to the Dispersion Element 104, and also likely the Dispersion Chamber 105. This connection can be “direct” such as by locating the Dispersion Element 104 and/or the Dispersion Chamber 105 within the Dispersion Reservoir 106 (partially or wholly), or the Dispersion Reservoir 106 could be located remotely from the Dispersion Element 104 (and potentially, the Dispersion Chamber 105), and connected by any appropriate means, such as a hose, tube, or wick, etc., channel way, canal, pump, gravity fed, absorption, electronic means, etc. As such, the purpose of the Distribution Reservoir 106, is to feed and replenish the Dispersion Element 104 with a supply (continual or periodic) of the desired fragrance(s) 100 to be dispersed. Although FIG. 4 illustrates a “straight line” type FDS 103, different sizes and shapes are possible as previously mentioned, including (but not limited to) an inverted T type FDS 103 with the vertical element serving as the Dispersion Chamber 105 containing the Dispersion Element 104, while the horizontal element could serve as the Dispersion Reservoir 106. The top of this Dispersion Chamber 105 could also contain a Dispersion Chamber Cap 107 in order to provide a convenient way to refill the Dispersion Reservoir 106 and/or the Dispersion Element 104.

Any FDS could potentially include a Dispersion Chamber Cap 107 (or potentially, multiple Dispersion Chamber Caps, not shown), to keep the Dispersion Element 104 and/or the fragrance 100 within the Dispersion Chamber 105 while the fragrance(s) are being dispersed. The Dispersion Cap(s) 107 can be threaded, pressed, screwed, twisted and/or locked onto the Dispersion Chamber 105, or attached by a variety of other means. The Dispersion Cap(s) 107 are generally removable, to allow for refilling of the Dispersion Reservoir 106 and/or Dispersion Element 104 with fragrance 100, or periodic replacement of the Dispersion Element 104, due to its end of life. Alternatively, the Dispersion Chamber Cap(s) 107 could also be removed and then replaced if a different fragrance(s) is desired to be dispersed (potentially also changing the Dispersion Element 104). Although not illustrated, a supplemental (or different) Dispersion Chamber Cap could be included at a different location (such as on the Dispersion Reservoir 106, as one example) in particular, if the Dispersion Reservoir 106 is remotely located from the Dispersion Chamber 105 and/or Dispersion Element 104, and/or the Dispersion Reservoir 106 is oriented differently than illustrated.

The FDS 103 can be permanently attached to or built into the artwork 102, or it may be fully removable, or partially removable. One such implementation of partial removability would be such that the FDS 103 is held in place with clips (much like AGC type fuse type clips, by but one example), to allow for easy removal and replacement of all or part of the FDS 103. Many other mounting options are possible for more permanently mounted FDS's, such as screws, rivets, tacks, tracks, adhesives or physically contained within the artwork 102 and/or the FDS 103 itself and removable, such as Velcro, clips, latches, catches and press fits of all sorts and sizes, semi-permanent adhesives, “screw on” type mounts, pressure, vacuum or magnetic mounts, etc., etc. An implementation using a track system, similar to track lighting, could also be used to provide for variable positioning of the FDS(s) 103 relative to the artwork 102, horizontally, vertically, or otherwise, such that a common frame could be re-used for future artwork, where the desired positioning of the FDS(s) 103 may be different for different artwork 102.

One such possible variation on the above FDS mounting systems of the second embodiment, would be to have the FDS 103, in whole or in part, removable and replaceable, such that the FDS 103 could be easily removed and replaced, either when all the fragrance 100 has been used, or when a different fragrance 100 is desired. This could include replacement cartridges, containing some or all of the components of FDS 103, that could be commercially available and prepackaged with various fragrance(s) 100, and could be either disposable or recyclable/reusable.

It should also be noted that multiple FDS devices 103 could be included as part of the second embodiment, at different or the same locations of the artwork 102 (may be or may not be, related to specific locations of the artwork 102 itself), to allow for dispersion of multiple fragrances 100. Alternatively, multiple fragrances 100 could be placed within a single FDS device 103, to provide for the dispersion of multiple fragrances 100.

An alternative mounting of the FDS device(s) 103 could be such that it provides fragrance 100 directly to the artwork 102. One such example of this would be if the Dispersion Element 104 of the FDS 103 is actually part of the artwork 102 itself. For example, the canvas of the artwork 102 could be directly connected (physically, or through tubing or a channel, drip or otherwise) to the FDS 103 or the Dispersion Reservoir 106, such that the fragrance(s) 100 are fed directly to the canvas and dispersed directly from the canvas. In this example, the canvas would serve as the Dispersion Element 104. Another such example could provide for a specific location(s) in the artwork to be fed with fragrance(s) 100 from an FDS 103, either at the canvas or some other specific part of the artwork 100 (such as a cloud made out of cotton, by but one such example, serving as the fragrance Dispersion Element 104, and used to disperse a fragrance) 100. Many other such variations are possible.

### Third Embodiment

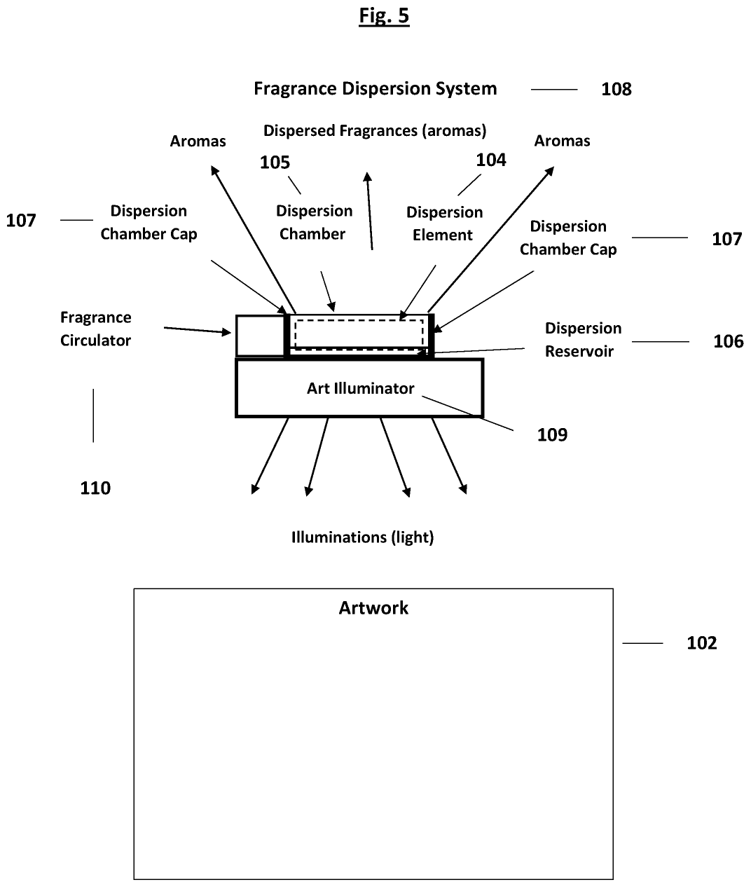

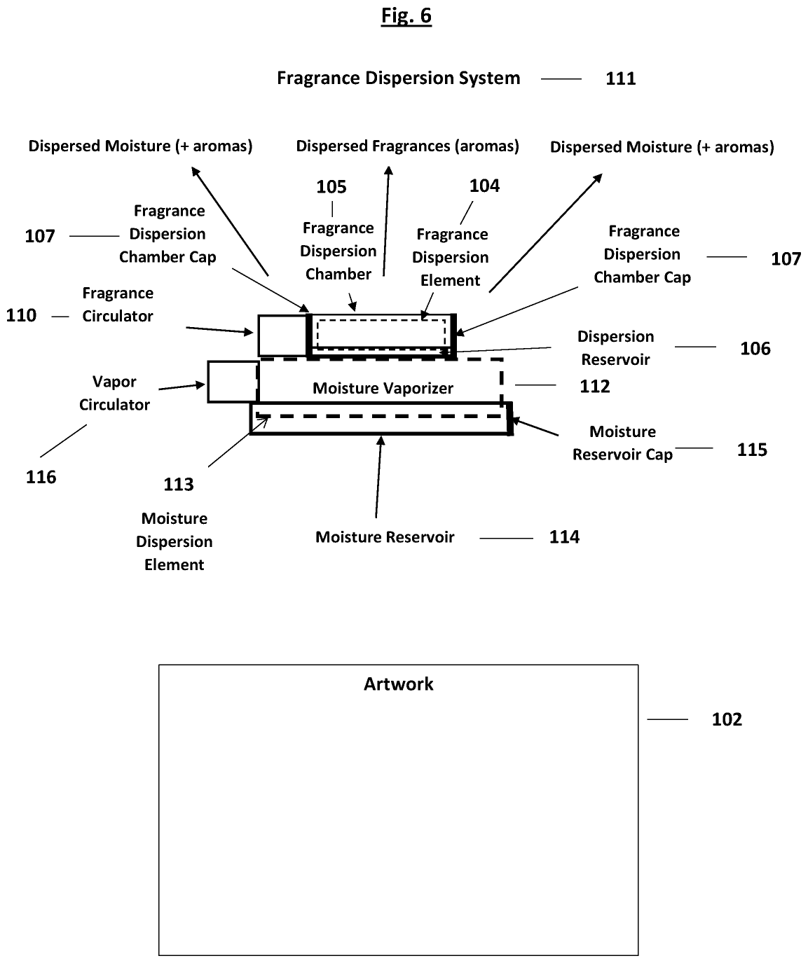

The third embodiment of the invention provides for an FDS 103, external from the artwork 102 itself. This could include any or all of the FDS 103 variations detailed in the second embodiment above, referencing a device similar to FIG. 4, but which is installed remotely from or adjacent to the artwork 102, or the third embodiment could include an FDS 108 integrated with an “artwork accessory”, such as a light, fan, vaporizer, plaque or signage, etc., also placed separate from or on the artwork 102. FIG. 5 details one such FDS 108 integrated into an artwork accessory, in this case, an art illuminator. While this is not the only possible option for integrating an FDS with an artwork accessory, it provides dual functionality to the artwork 102, namely, proving both illumination as well as fragrance 100 to the artwork 102.

The main elements of this such possible third embodiments, integrated into an art illumination device, are as detailed in FIG. 5, and include an FDS 108 similar to the FDS 103 in FIG. 4, resized and/or reshaped to be located adjacent to, with, on, or in an art illuminator 109, such as a light or LED device. The added benefit of combining the art illuminator 109 with the FDS 108, is that all lights/LEDs generate some level of supplemental heat. As this heat is generated, air flows upwards, in and around the FDS 108. This airflow provides added circulation of the fragrance(s) 100 from the FDS 108. The light/LED or art illuminator 109 could also provide heating of the Dispersion Reservoir 106, which also improves dispersion of the fragrance(s) 100, by raising the temperature of the fragrance(s) 100. It should be noted that that the FDS 108 in FIG. 5 could be located above, below, or in any other relation to the artwork 102. The dispersed fragrances, or aromas are as indicated in FIG. 5.

Although not shown, such light or LED could be battery, solar, or AC/DC powered. Also shown in FIG. 5, is a Fragrance Circulator 110, such as a fan or other thermal transfer device, for providing additional and increased circulation of the fragrance(s) 100 from the FDS 108 towards and/or near the desired artwork 102. Such a circulation device could be located aside of, on top of, underneath, behind, around or otherwise adjacent to the FDS 108, and also as potentially integrated within the third embodiment, to provide for increased dispersion of the fragrance(s). Although a single Fragrance Circulator 110 is shown, there could be multiple Fragrance Circulators in and/or around the FDS 108, to further improve circulation of the fragrance(s) 100. These Fragrance Circulator(s) 110 could be battery, solar, or AC/DC powered (also not shown). Any of the above Fragrance Circulator(s) 110 could also be integrated with the FDS 103 of the second embodiment.

Another such possible integrated FDS of the third embodiment would be to combine artwork 102 with an FDS with a vaporizer, to assist in the dispersion of the fragrance(s). This integration is highlighted in FIG. 6, and is similar to FIG. 5, except that the Art Illuminator 109 is now replaced with a Moisture Vaporizer 112. The Moisture Vaporizer 112 includes holes/perforations as indicated, such that the moisture contained within it is released through evaporation, vaporization, ultrasonic vibration, atomization, etc., into the nearby environment of the artwork. The moisture is also replenished from these same or similar holes/perforations via absorption, osmosis, convection, gravity, pump, pressure differential, etc., from the Moisture Reservoir 114. Moisture Vaporizer 112 could also optionally contain a Moisture Dispersion Element 113, much like the Fragrance Dispersion Element 104, to hold the moisture while it is being dispersed, including absorbing replenishment moisture from the Moisture Reservoir 114. Either (or both) the Moisture Vaporizer 112 or the Moisture Reservoir 114 would also likely include an optional Cap(s) 115 as indicated, to provide for the moisture (water or otherwise) contained within the Moisture Reservoir 114 to be conveniently replenished.

Moisture is released from the Moisture Vaporizer 112 as indicated in FIG. 6, and inherently combines with the dispersed fragrance(s) being released from the FDS 111, improving the fragrance dispersion. This dispersion of either/both the moisture and/or fragrance(s) can be further improved with the addition of one (or more) Vapor Circulators 116 and/or Fragrance Circulators 110, as also shown in FIG. 6. These optional circulators can be powered, by battery, AC/DC or solar powered (not shown).

Alternatively or in addition to, the fragrance(s) 100 can be added directly to the Moisture Reservoir 114 and/or the Moisture Dispersion Element 113 mentioned previously, such that the Fragrance Dispersion Element 104, Fragrance Dispersion Chamber 105 and Fragrance Dispersion Chamber Cap(s) 107 in FIG. 6 can be optionally eliminated altogether in this third embodiment, by virtue of the moisture (and therefore fragrance) dispersion from the Moisture Vaporizer 112.

It should be noted that the third embodiment can be enabled to have one or multiple components (FDS, Illumination, Moisture, etc.), or any combination thereof. It should also be noted that in any of the above embodiments, some of the components might be eliminated, while still providing the novelty of the invention. By way of one example, if only the Fragrance Dispersion Element is present in any of the above embodiments, the basic novelty of the invention is still provided.

1. A system for providing fragrance to an artwork, the system comprising:
a) a fragrance dispersion system configured to disperse a fragrance;
b) wherein the fragrance dispersion system is configured to be positioned adjacent to, within, or on an artwork.

2. The system of claim 1 wherein the fragrance dispersion system is positioned on or within the body of a frame for holding the artwork.

3. The system of claim 1 wherein the fragrance dispersion system comprises a fragrance additive integrated with materials used to create the artwork, the scent of the fragrance additive being related to what is depicted in the artwork.

4. The system of claim 3 wherein the fragrance additive forms part of a paint in the artwork.

5. The system of claim 1 wherein the fragrance dispersion system comprises a fragrance dispersion element that is replaceable.

6. The system of claim 1 wherein the fragrance dispersion system is detachable from the artwork.

7. The system of claim 1 wherein the fragrance dispersion system comprises a fragrance dispersion chamber for receiving a replaceable chamber dispersion element.

8. The system of claim 1 wherein the fragrance dispersion system comprises a fragrance dispersion reservoir for holding a source of the fragrance.

9. The system of claim 1 wherein the fragrance dispersion system comprises a cap for containing a source of the fragrance.

10. The system of claim 1 wherein the fragrance dispersion system comprises a fragrance circulator for circulating the fragrance in or around the artwork.

11. The system of claim 10 wherein the fragrance circulator is a fan.

12. The system of claim 10 wherein the fragrance circulator is a light.

13. The system of claim 1 wherein the fragrance dispersion system is configured to be attached to an illuminator for illuminating the artwork.

14. The system of claim 1 wherein the fragrance dispersion system forms part of an illuminator for illuminating the artwork.

15. A fragrance dispersion apparatus comprising:
a) a fragrance dispersion system configured to be positioned adjacent to, within, or on an artwork;
b) wherein the fragrance dispersion system is configured to disperse a fragrance in or around the artwork.

16. The apparatus of claim 15 wherein the fragrance dispersion system is positioned on or within the body of a frame for holding the artwork.

17. The system of claim 15 wherein the fragrance dispersion system comprises a fragrance dispersion element that is replaceable.

18. The system of claim 15 wherein the fragrance dispersion system further comprises a fragrance dispersion chamber for receiving a replaceable chamber dispersion element.

19. A method of providing fragrance to an artwork, the method comprising:
a) providing a fragrance dispersion system configured to disperse a fragrance;
b) positioning the fragrance dispersion system adjacent to, within, or on an artwork.

20. The method of claim 19 wherein the fragrance dispersion system is positioned on or within the body of a frame for holding the artwork.
